(19) United States
(12) Patent Application Publication
(10) Pub. No.: US 2007/0098828 A1
(43) Pub. Date: May 3, 2007

(54) Methods and compositions for the prevention and regression of neovascularization

(76) Inventor: Lucia Irene Gonzalez-Villasenor, Baltimore, MD (US)

Correspondence Address: Thomas M. Saunders;Brown Rudnick Berlack Israels LLP, BOX IP, One Financial Center, Boston, MA, 02111, US

(21) Appl. No.: 11/589,460

(22) Filed: Oct. 30, 2006

Related U.S. Application Data
(60) Provisional application No. 60/732,266, filed on Nov. 1, 2005.

(51) Int. Cl. A61K 35/74

(52) U.S. Cl. 424/780

(57) ABSTRACT

The present invention provides methods and compositions for the prevention and treatment of conditions characterized by neovascularization. Preferably, the present invention is directed to a potent inhibitor of angiogenesis. This inhibitor may be useful for the treatment of ocular diseases by preventing neovascularization. The invention is based, at least in part, on the isolation of the inhibitor of angiogenesis from, inter alia, non-processed intracellular extracts from the bacteria Escherichia coli. The present invention also includes a method of treating a condition characterized by neovascularization in a mammal by administration of the inhibitor of angiogenesis, wherein the treatment shows prevention and/or regression of the condition. Such regression of the condition may be, for example, to inhibit aberrant angiogenesis in the eye or to inhibit the growth of carcinogenic tumors. Examples of diseases may include, but are not limited to, ocular diseases and cancer.

### CROSS REFERENCE TO RELATED APPLICATIONS

This Application claims the benefit of U.S. Provisional Application No. 60/732,266, filed on Nov. 1, 2005. The entire teachings of the above application is incorporated herein by reference.

### BACKGROUND OF THE INVENTION

Angiogenesis involves the migration of vascular endothelial cells into tissue in response to specific signals and involves a complex process characterized by infiltration of the basal lamina by vascular endothelial cells in response to angiogenic growth signal(s), migration of the endothelial cells toward the source of the signal(s), and subsequent proliferation and formation of the capillary tube, or new blood vessels. Typically, these molecules appear to maintain the microvasculature without capillary growth for prolonged periods of time. Although angiogenesis is a highly regulated process under normal conditions, many diseases are driven by persistent unregulated angiogenesis. In such disease states, unregulated angiogenesis can cause a particular disease directly or exacerbate an existing pathological condition, for example, in ocular diseases and cancer.

The growth and metastasis of solid tumors are angiogenesis-dependent. Tumors obtain their own blood supply by inducing the growth of new capillary blood vessels. Neovascularization of a tumor enables the metastatic spread of aggressive tumor cells by providing a route of escape for the metastatic cells. They metastasize to distant sites, such as liver, lung or bone, as well as nurture the tumor by providing nutrients and growth factors essential for tumor growth. Inhibitors of angiogenesis can regress or prevent the growth of tumors. Although several angiogenesis inhibitors are under development for treating angiogenic diseases, they have shown to have different antiangiogenic effects as well as have unwanted side-effects. Some compounds are very potent but may cause systemic toxicity in humans at high doses and others have weak antiangiogenesis effects.

Additionally, ocular neovascularization has been implicated as the most common cause of blindness and underlies the pathology of many diseases of the eye including diabetic retinopathy and age-related macular degeneration. These diseases are characterized by aberrant choroidal neovascularization (CNV) of the retinal vasculature causing hemorrhage, tissue damage and retinal scarring. The only current effective therapy for advanced ocular disease is laser photocoagulation in which the effect of treatment is temporary due to the high rate of reoccurrence. Plus, many patients who develop CNV are no longer available for laser therapy because CNV is too large for laser treatment or the location cannot be determined for accurate aim of the laser.

Accordingly, there remains a need for discovering new inhibitors of angiogenesis that are more target-specific, are more potent and have fewer side-effects.

### SUMMARY OF THE INVENTION

The present invention provides compositions and methods for the prevention and treatment of conditions characterized by neovascularization. The inhibitor of the present invention can prevent and inhibit aberrant neovascularization such as those observed in cancer and ocular diseases. The invention is based, at least in part, on the isolation of the inhibitor of angiogenesis from, inter alia, non-processed intracellular extracts from the bacteria Escherichia coli. The inhibitor of angiogenesis may also be present in other organism sources, or may have homologous peptide, protein or enzyme sequences and/or similar polysaccharides, oligonucleotide and DNA sequences and/or chemical structures similar to isoprenoid precursors, and/or small molecules such as steroids, pyrimidine, or quinoline, and they may be present in other eukaryotic systems such as yeast and mammals including humans. As described below, the inhibitor of angiogenesis of the present invention strongly inhibits the growth of mammalian derived microvessel endothelial cells in vitro and the progression of induced neovascularization in retina tissue in vivo. The inhibitor of angiogenesis may be, but is not limited to, a peptide, protein, enzyme, polysaccharide, oligonucleotide, DNA, pigment precursor isoprenoid and/or a small molecule such as a steroid, pyrimidine or quinoline.

Additionally, the present invention is directed to a method of preventing or inhibiting neovascularization in a mammal, wherein the method shows a regression of neovascularization. The present invention is also directed to a method of treating a condition characterized by neovascularization in a mammal, wherein the method includes administering to the mammal a therapeutically effective amount of an inhibitor of angiogenesis, wherein the treatment shows prevention and/or regression of neovascularization. Examples of a diseases characterized by neovascularization include, but are not limited to, cancer, ocular diseases such as diabetic retinopathy or age-related macular degeneration, retinitis pigmentosa, hemangioma, arthritis, psoriasis, angiofibroma, delayed wound healing, granulations, hemophilic joints, hypertrophic scars, non-union fractures, Osler-Weber syndrome, pyogenic granuloma, scleroderma, trachoma, and vascular adhesions.

### BRIEF DESCRIPTION OF THE FIGURES

Other features and advantages of the invention will be apparent from the following description of the preferred embodiments thereof and from the claims, taken in conjunction with the accompanying drawings, in which:

FIG. 1A shows a plot of four-fold serial dilutions of purified recombinant Troponin I (rTnI) on gel filtration (S-200) column chromatography vs. optical density (OD) using a Bovine Lung Microvessel endothelial cell (BLM EC) in vitro assay. A colorimetric reaction based on the endogenous activity of Acid Phosphatase (AP) expressed in OD units was recorded at 410 nm for each dilution using a standard spectrophotometer. Maximum inhibition of BLM ECs is observed in the fraction containing E. coli proteins (a). Less activity was observed in fractions containing purified rTnI from two Sephacryl S-200 runs (b and c), and no activity was observed in purified MDP-14 used as independent protein control (d) or vehicle control (e). Purified rTnI is oligomeric in solution and is associated with traces of the inhibitor of angiogenesis which apparently co-purifies with rTnI to a limited extent;

FIG. 1B shows a spectrophotometer plot of four-fold serial dilution of MDP-14 at various stages of purification on anion exchange (DEAE) and gel filtration (S-100) column chromatography vs. optical density (OD) using a Bovine Lung Microvessel endothelial cell (BLMEC) in vitro assay. A colorimetric reaction based on the endogenous activity of Acid Phosphatase (AP) expressed in OD units was recorded at 410 nm for each dilution using a standard spectrophotometer-(A) MDP-14 raw extract; (B) flow through fraction from DEAE; (C) MDP-14 eluted from DEAE; (D) MDP-14 after S-100; (E) purified MDP-14; (F) vehicle control (dilution buffer);

FIGS. 2A-2D are fluorescent microscopic images of a sample mouse retina showing prevention of choroidal neovascularization with 20 μg (FIG. 2A) and 38 μg (FIG. 2C) total of the inhibitor of angiogenesis administered by intravitreous injections;

FIG. 2E shows a graphic representation of the average size of the CNV area in mm²×10⁻³ for the prevention studies with intravenous injections in treated (20 μg total dosage at N=29) and untreated (control at N=26) mice retina;

FIG. 2F shows a graphic representation of the average size of the CNV area in mm²×10⁻³ for the prevention studies with intravenous injections in treated (38 μg total dosage at N=26) and untreated (control at N=26) mice retina;

FIGS. 3A-3D are fluorescent microscopic images of a sample mouse retina showing prevention of choroidal neovascularization with 28 μg (FIG. 3A) and 53 μg (FIG. 3C) of the inhibitor of angiogenesis administered by periocular injections;

FIG. 3E shows a graphic representation of the average size of the CNV area in mm²×10⁻³ for the prevention studies with periocular injections in treated (28 μg dosage at N=26) and untreated (control at N=26) mice retina;

FIG. 3F shows a graphic representation of the average size of the CNV area in mm²×10⁻³ for the prevention studies with periocular injections in treated 53 μg dosage at N=30) and untreated (control at N=26) mice retina;

FIGS. 4A-4C are fluorescent microscopic images of a sample mouse retina showing regression of CNV from a baseline (FIG. 4A) to a dosage of the inhibitor of angiogenesis (FIG. 4B) to a control (FIG. 4C) administered by intravitreous injections;

FIG. 4D is a graph representing the increase of CNV area in mm²×10⁻³ after laser treatment from day 0 to day 7, and then the decrease of CNV area in mm²×10⁻³ from day 7 through 14 in the treated (53 μg total at N=29) and untreated (control at N=29) mice retina; and

FIG. 5 is an analysis of Sephacryl S-100 gel filtration fractions by Coomassie stained, reduced SDS-PAGE. Strong potency in the BLM in vitro assay was associated with fractions with stained proteins between 7 and 70 KDa-(a) Protein Marker, (b) through (f) fractions with different MW proteins.

### DETAILED DESCRIPTION OF THE INVENTION

The potent inhibitor of angiogenesis of the present invention was discovered in bacteria Escherichia coli while producing recombinant polypeptides for screening inhibitory activity in an in vitro endothelial cell assay using human and bovine lung microvessels (HLM and BLM). We have shown in mice that both prevention and regression of induced choroidal neovascularization, a collection of blood vessels embedded within a fibrous tissue that provide nutrition to the retina, is induced using preparations containing the novel inhibitor of angiogenesis.

The invention is based, at least in part, on the isolation and characterization of the inhibitor of angiogenesis bacterial extracts and a method of preventing and decreasing aberrant neovascularization such as that observed in cancer and ocular diseases using the inhibitor of angiogenesis. According to the present invention, the inhibitor of angiogenesis significantly inhibits, for example, laser-induced choroidal neovascularization (CNV) in mouse retina, in vivo, and specifically inhibits microvessel endothelial cell growth in vitro in bovine and human lung microvessel (BLM and HLM) cell lines. Furthermore, this inhibitor of angiogenesis found in fractions containing proteins has a molecular weight in the range of about 7 to about 70 KDa and a yellowish-pinkish in color. In addition, the specific and gradual mammalian endothelial cell growth inhibition and cell death observed in vitro seems to suggest a mechanism of apoptosis and not induced toxicity. Lack of generalized toxicity is demonstrated by the survival of non-ECs present in the BLM cells, which are not a pure cell line. We also believe that the complete suppression of endothelial cell growth in vitro may imply that the inhibitor of angiogenesis has anti-growth factor activity that is broader than β-FGF alone (Feldman & Rouleau (2002) Microvascular Research 63:41-49) or β-FGF and VEGF together.

The inventor of the present application has unexpectedly discovered that the inhibitor of angiogenesis of the invention causes strong inhibition of endothelial cells during a study of the biological activity of fractions derived from different stages of purification of the recombinant protein Troponin-I (rTnI) obtained by fermentation of the bacteria Escherichia coli. rTnI is claimed to have antiangiogenic activity according to Moses et al., U.S. Pat. No. 5,837,680 (also published in PNAS (1999) 96:2645-2650), the whole of which is hereby incorporated by reference.

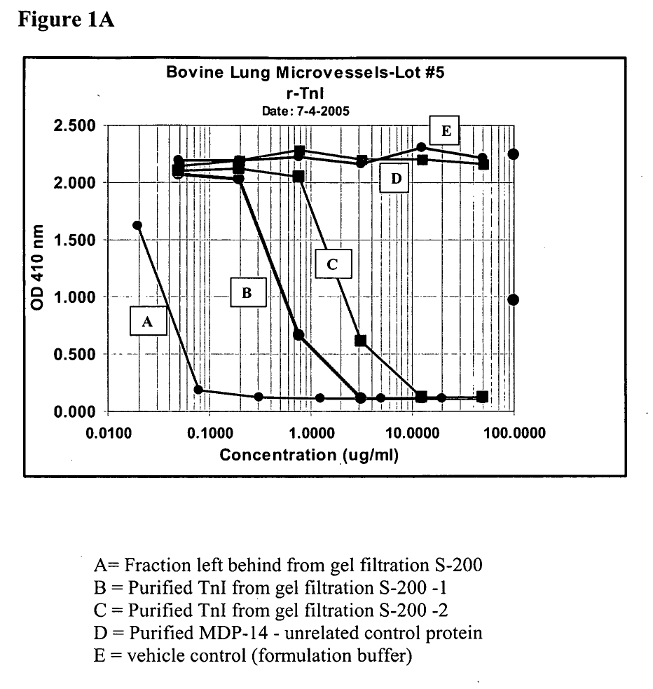

During the process of purifying and analyzing rTnI activity, it was observed that the biological activity of rTnI, as measured in refined Bovine or Human Lung microvessel endothelial assay, decreased inversely with increasing rTnI. In fact, some fractions containing E. coli proteins with only traces (picomolar) of rTnI had very strong potency (FIG. 1A). Furthermore, tests performed with or without rTnI fractions showed strong potency in the in vitro assay. This observation led to the conclusion that the activity observed in rTnI preparations could be entirely or partly due to an unidentified compound that partially co-purified with rTnI.

To determine whether another compound in E. coli might have BLM inhibitory activity, the test was performed using fractions derived from different stages of purification of a protein unrelated to rTnI called Macrophage Derived Protein-14 (MDP-14), also obtained by fermentation in E. coli (using a different purification procedure). MDP-14 was used as control for the in vitro assay. It was reasoned that purified MDP-14 should have no biological activity in the absence of E. coli proteins or intracellular compounds, but unpurified or partially purified MDP-14 containing E. coli proteins and intracellular compounds should show strong inhibition of endothelial cells in the in vitro assay.

Indeed, a potent inhibitory activity was observed in the unpurified fractions of MDP-14 and completely disappeared in fractions containing pure MDP-14. The different levels of potency observed in MDP-14 preparations was obtained in primary bovine lung microvessel endothelial cells plated and incubated at 37° C. for 72 hours in the presence of samples and controls. Cell numbers were estimated by a colorimetric reaction that measured endogenous Acid Phosphatase activity expressed as OD units. FIG. 1B shows representative data of serial dilutions of rat-MDP-14 in μg/ml at various stages of purification vs. Optical Density (OD) at 410 nm wavelength. OD values were inversely proportional to the potency of samples. It was determined that the lower the OD value, the stronger the cell inhibition and potency of samples (FIGS. 1B(a) and 1B(b)). High OD values in controls (FIGS. 1B(e) and 1B(f)) indicated no inhibition of cell-growth. In some samples (FIGS. 1B(c) and 1B(d)) cell inhibition decreased gradually (higher OD) as the sample was diluted. Each curve shown in FIGS. 1A and 1B is the average of testing each sample at least in four different plates with a minimum of four replicates for each point. Thus the value for each dilution represents the average of 16 points per sample.

These results not only unequivocally confirmed that there is a compound in E. coli extracts that causes the inhibitory activity but also implied that rTnI derived from bacterial extracts do not show inhibitory activity when all of the E. Coli proteins and other bacterial intracellular compounds are removed during purification.

In patients with an ocular disease, a reduction in CNV in the retina will result in diminished retinal damage and scarring, thereby preventing the loss of sight. In cancer patients, a reduction or inhibition of tumor growth will result in an increase in survival and quality of life. This process would be dependent on the extent of the spread of tumor and type of cancer. While there are more than sixty antiangiogenic compounds in clinical trials for different types of cancers, none of them have shown a significant positive effect in cancer patients when administered individually. However, in combination with radiotherapy or chemotherapy, a few antiangiogenic compounds have shown a modest rate of increase in patient survival. Nevertheless, such compounds have caused many adverse reactions in the patients treated. Therefore, more potent and less toxic antiangiogenic compounds are needed for effective treatment of ocular diseases and cancer. Most of the antiangiogenic compounds currently tested have been used at very high concentrations in animal studies and in vitro assays in order to observe a positive effect. The presently claimed antiangiogenic compound may be a more effective inhibitor of angiogenesis at many times lower dosages than the high doses used for other antiangiogenic compounds tested thus far.

Accordingly, the present invention is directed to a method of treating a condition characterized by neovascularization in a mammal, wherein the method comprises the steps of administering to the mammal a therapeutically effective amount of an inhibitor of angiogenesis, wherein the method shows prevention or regression of neovascularization. In one aspect, the mammal is an animal or a human. In another aspect, the therapeutically effective amount of an inhibitor is in a pharmaceutically acceptable carrier. The inhibitor of angiogenesis, for example, prevents and causes regression of neovascularization in retina but it is not limited to choroidal vessels in mammalian eyes.

The inhibitor of angiogenesis of the present invention is found in crude extracts of bacterial Escherichia coli and has a molecular weight in the range of about 7 to about 70 kDa. In one aspect, the inhibitor of angiogenesis specifically inhibits endothelial cells, for example, human and bovine lung microvessel endothelial cells (HLM-ECs and BLM-ECs).

The inhibitor of angiogenesis of the present invention may be, but is not limited to, a peptide, protein, polysaccharide, oligonucleotide, DNA, pigment precursor isoprenoid and/or a small molecule such as a steroid, pyrimidine or quinoline. The inhibitor of angiogenesis may be in the range of about 7 to 70 kDa. Preferably, the bacterial protein is in the range of about 7 to 10 kDa, in the range of about 10 to 20, in the range of about 20 to 30, in the range of about 30 to 40, in the range of about 40-50, in the range of about 50-60, in the range of about 60-70, in the range of about 10 to 15, in the range of about 15 to 20, in the range of about 20 to 25, in the range of about 25 to 30, in the range of about 30 to 35, in the range of about 35 to 40, in the range of about 40 to 45, in the range of about 45 to 50, in the range of about 50-55, in the range of about 55 to 60, in the range of about 60-65, or in the range of about 65 to 70. The term “about” will be understood to include any value above and below the given number.

In one aspect of the invention, some of the biologically active fractions containing a complex pattern of bacterial proteins also show a range of colors from yellowish to greenish to pinkish which are common bacterial pigments. The inhibitor of angiogenesis of the invention may include a small compound such as a pigment precursor or a polysaccharide that is present in the bacterial extract which causes the strong inhibition of cell growth. E. coli lacks the universal carotenoid starting material phytoene but synthesizes isophentyl pyrophosphate the key metabolite in terpenoid formation which is important in the isoprenoid biosynthetic pathway that leads to the synthesis of carotenoids (Vitamin A) in higher organisms. This compound plays a role in the prevention of cancer and is essential for the visual system. In another aspect of the invention, an isoprenoid precursor may be present in the E. coli extract and may be the compound causing the strong cell inhibition observed in the in vitro assay as described below, and the regression of choroidal neovascularization in vivo in mice.

As used herein, “neovascularization” will be understood to include abnormal neovascularization, including the formation of new blood vessels, larger blood vessels, more branched blood vessels, and any and all mechanisms that lead to inappropriate or increased blood carrying capacity to a diseased tissue or site. The inhibitor of angiogenesis of the present invention will be understood to prevent or diminish abnormal neovascularization, irrespective of the actual mechanism of action.

Also as used herein, “cancer” refers to a cellular tumor. Cancer cells have the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type, or stage of invasiveness. Examples of cancers include, but are not limited to, carcinoma and sarcoma such as leukemia, sarcomas, osteosarcoma, lymphomas, melanoma, ovarian cancer, skin cancer, testicular cancer, gastric cancer, pancreatic cancer, renal cancer, breast cancer, prostate cancer, colorectal cancer, cancer of the head and neck, brain cancer, esophageal cancer, bladder cancer, adrenal cortical cancer, lung cancer, bronchus cancer, endometrial cancer, nasopharyngeal cancer, cervical or hepatic cancer, or cancer of unknown primary site. In addition, cancer can be associated with a drug resistance phenotype.

“Ocular diseases” are associated with neovascularization which can be treated with the inhibitor of angiogenesis of the present invention and may include, but are not limited to, diabetic retinopathy, age-related macular degeneration, retinitis pigmentosa, neovascular glaucoma, retinoblastoma, retrolental fibroplasias, uveitis, retinopathy of prematurity, corneal graft neovascularization as well as other eye inflammatory diseases, ocular tumors and diseases associated with choroidal or iris neovascularization. See, e.g., reviews by Waltman et al. (1978) Am. J. Ophthal. 85:704-710; and Gartner et al. (1978) Surv. Ophthal. 22:291-312.

Other disorders which can be treated with the inhibitor of angiogenesis of the present invention include, but are not limited to, hemangioma, arthritis, psoriasis, angiofibroma, atherosclerotic plaques, delayed wound healing, granulations, hemophilic joints, hypertrophic scars, non-union fractures, Osler-Weber syndrome, pyogenic granuloma, scleroderma, trachoma, and vascular adhesions.

The term “therapeutically effective amount” will be understood to mean an amount of the inhibitor of angiogenesis that prevents or treats diseases characterized by neovascularization upon administration to a mammal, including human. The amounts may be effective, for example, to slow the growth of a tumor, to stop the growth of a tumor, or to induce tumor regression or remission upon administration to the mammal. Such effects are achieved while exhibiting negligible or manageable adverse side effects on normal, healthy tissues of the mammal. The “therapeutically effective amount” can vary from mammal to mammal or patient to patient, depending on a number of factors including, but not limited to, the extent of disease and the size of the mammal.

The present invention also provides pharmaceutical compositions. Such compositions comprise a therapeutically effective amount of an inhibitor of angiogenesis of the present invention, and a pharmaceutically acceptable carrier.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. The parental preparation can be enclosed in ampoules, disposable syringes or multiple dose vials made of glass or plastic.

These compositions can take the form of solutions, suspensions, emulsion, tablets, pills, capsules, powders, sustained-release formulations and the like. The composition can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the inhibitor of angiogenesis of the present invention, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the inhibitor of angiogenesis of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test bioassays or systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The following additional examples are presented to illustrate the advantages of the present invention and to assist one of ordinary skill in making and using the same. These examples are not intended in any way otherwise to limit the scope of the disclosure.

### EXAMPLE I

### Isolation of the Inhibitor of Angiogenesis of the Present Invention

The inhibitor of angiogenesis of the present invention has been partially isolated from fractions containing E. Coli proteins during the purification of recombinant Troponin-I (rTnI) and purification of MDP-14 using standard chromatography purification procedures. Such activity has been also observed in E. Coli extracts without a recombinant protein when tested in the BLM in vitro assay. In the case of E. coli extracts with rTnI, briefly, the process consists of preparing a raw E. coli extract from solubilized inclusion bodies and purifying rTnI by a four-step chromatography procedure entailing anion and cation exchange chromatography on Sartobind filters, and metal binding column chromatography on nickel Sepharose and gel filtration chromatography on Sephacryl column. Alternatively, fractions containing the unidentified compound have been isolated from E. coli extracts containing soluble bacterial proteins such as MDP-14. The purification of MDP-14 consists of two column chromatography steps entailing anion-exchange on DEAE Sepharose and gel filtration on Sephacryl S-100 media. (See, for example, Protein Purification Handbook, Protein Amplification and Single Purification, Amersham Pharmacia Biotech, Edition AB, (2001), page 110; and Protein Purification Protocols, Ed. Shawn Doonan. Methods in Molecular Biology, Vol. 59. Humana Press, Totowa, N.J., page 405, for general protein purification and biochemical methods.)

### EXAMPLE II

### Purification and Characterization of the Inhibitor of Angiogenesis

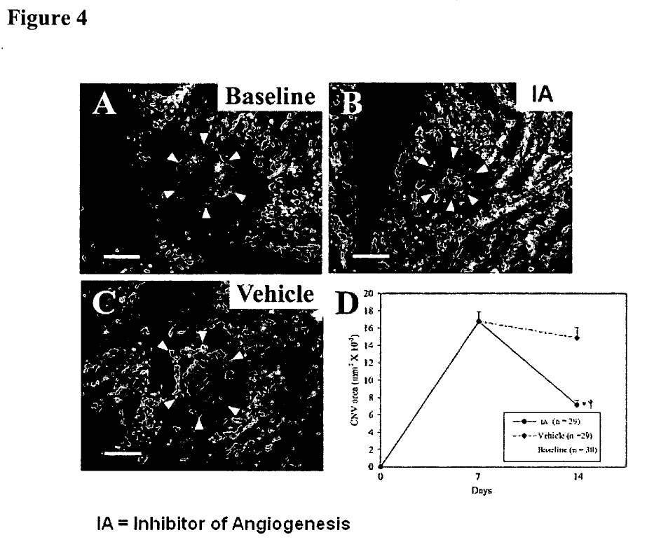

Fractions containing MDP-14 and E. coli proteins were eluted from a weak anion exchange DEAE Sepharose column followed by gel filtration chromatography on Sephacryl S-100 using an automated bioprocess system AKTA Explorer and the Unicorn software of Amersham. The fractions were collected using a fraction collector attached to the bioprocess system with standard 15 ml tubes. Based on further electrophoresis analysis known in the art, the inhibitor of angiogenesis was detected in fractions containing proteins in the size range of 7 to 70 KDa which have several physicochemical and biological properties including molecular weight, isoelectric point, hydrophobicity, oxidizing and reducing cysteine residues, carboxy and amino termini, electric charge by native and a particular molecular size by denatured electrophoresis, purity by gel filtration and electrophoresis, level of endotoxins and DNA, amino acid sequence analysis to identify the protein, analysis of its molecular structure including magnetic resonance, and light scattering ultracentrifugation for analysis of aggregates and stability studies at different temperatures. Based on strong biological activity present in protein fractions analyzed by electrophoresis in denatured SDS gels, it has been observed in SDS-gels that the inhibitor of angiogenesis may be a bacterial protein in the range of 7 to 70 KDa (FIG. 5). These proteins remain in the fractions left behind during MDP-14 purification but are not present in purified MDP-14 which is 14 KDa in size, has a purity >99% and has no biological activity in the in vitro BLM assay (FIG. 1F).

While further isolation and identification of the inhibitor of angiogenesis can be performed using the BLM assay described in the present invention, other methods known in the art can be used to further characterize the inhibitor of angiogenesis. The isolation methods can be modified as needed to yield fractions that are compatible with the bioassay. In addition to the bioassay, standard physico-chemical analysis is performed to identify the general nature (protein, lipid, etc) of the materials as well as to obtain detailed information about the molecular composition. For example, High Pressure Liquid Chromatography coupled with mass spectrometers is a very powerful tool for analysis of most biochemical compounds. As the properties become more defined, it is appreciated that more selective and efficient methods can be used. An ordinary skilled artisan can follow several different isolation and characterization methods as practiced in the art; for example, pigments are described in Zhu et al., J. Agric. Food Chem. (Jul. 13, 2005) 53(14):5593-7, Yang et al., J. Proteome Res. (May-June 2005) 4(3):846-54, and Ke et al., Arch. Biochem. Biophys. (Apr. 1, 2005) 436(1):110-20; quinines can be characterized using the methods as described in Paddock et al., Biochemistry (May 10, 2005) 44(18):6920-8; polysaccharides can be characterized using the methods described in FEMS Microbiol. Lett. (Dec. 15, 1998) 169(2):283-7.

The MS method comprised digesting with trypsin gel bands that were cut out from the electrophoresis gels. The extracted peptides were then analyzed by reverse phase nano-LC-MS/MS on a Finnigan LTQ mass spectrometer. The raw data were searched against the SwissProt database with assumptions for trypsin using the Thermo Finnigan Turbo Sequest software. The analysis provided protein names and hits along with the specific peptides. The statistical significance of each peptide was scored and summed to give the protein score. The highest scoring proteins were the most reliable hits. The results were then filtered based on score cut-off's for singly, doubly, and triply charged peptides. The data were also filtered to only show unique peptides. The following three hits were selected: Murein Lipoprotein, Lumazine Synthase and the Cold shock protein cspA and/or cspC.

### EXAMPLE III

### Induced Choroidal Neovascularization

Purified rTnI containing small quantities of the unidentified compound was used to demonstrate inhibition of microvessel endothelial cell growth and to carry out experiments in vivo in mice. Specifically, a laser injury rodent model was used for the assessment of angiostatic agents for potential use in the treatment of age-related macular degeneration (AMD).

Laser-induced choroidal neovascularization (CNV) was achieved by a modification of a known technique. (Tobe, et al., “Targeted Disruption of the FGF2 Gene Does Not Prevent Choroidal Neovascularization in a Murine Model.” Amer. J. Pathol. (November 1998) 153(5):1641-1646.) Briefly, 4 to 5 week old male C57BU6J mice were anesthetized with ketamine hydrochloride (100 mg/kg body weight) and the pupils were dilated with 1% tropicamide. Three burns of krypton laser photocoagulation (100 pm spot size, 0.1 seconds duration, 150 mW) were delivered to each retina using the slit lamp delivery system of a Coherent Model 920 Photocoagulator and a hand held cover slide as a contact lens. Burns were performed in the 9, 12, and 3 o'clock positions of the posterior pole of the retina. Production of a bubble at the time of laser, which indicates rupture of Bruch's membrane, is an important factor in obtaining CNV. Mice were then randomly assigned to treatment. For prevention studies with intravitreous injections, mice (N=29/experiment) were treated with 5 μL (10 μg and 19 μg) of rTnI containing inhibitor of angiogenesis (IA) or vehicle control at day 0. A second intravitreous injection with 5 μL (19 μg and 38 μg) rTnI containing inhibitor of angiogenesis or vehicle control was administered at day 7. For prevention studies with periocular injections, mice (N=29/experiment) were treated daily with 1 μl (2.0 μg and 3.8 μg) rTnI or vehicle control from day 0 through 14. For intravitreous injections, mice were killed at day 14, and for periocular injections mice were killed at day 15, with an overdose of pentobarbital sodium, and their eyes were rapidly removed and frozen in optimal cutting temperature embedding compound (OCT). The analysis of induced CNV was performed at the end of each treatment in anesthetized mice by removing the cornea and lens and dissecting from the eyecup the entire retina which is flat mounted. The flat mounts were examined by fluorescence microscopy and the images were digitized using a three color video camera. The total area of CNV associated with each burn with respect to treatment group was measured in mm²×10⁻³. Statistical comparisons were made between the size of lesions in mice treated versus un-treated animals by two-tailed t-test.

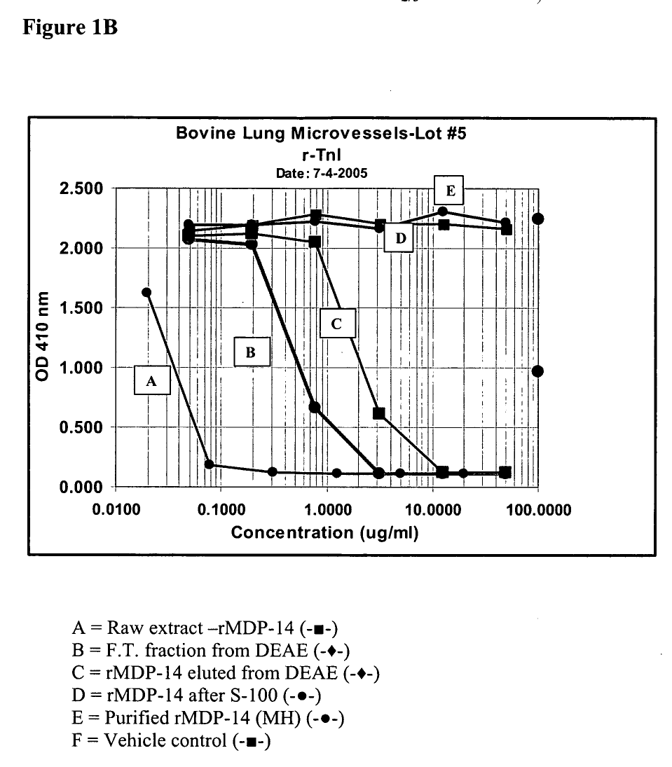

The development, prevention and regression of CNV was evaluated and demonstrated, as shown in FIGS. 2, 3 and 4.

FIGS. 2A-2F are results of experiments performed to show the prevention of choroidal neovascularization. Preparations of recombinant Troponin I (rTnI) containing the inhibitor of angiogenesis (IA), were administered once a week for 2 weeks with a total dose of 20 μg and 38 μg. The CNV lesion was analyzed by fluorescence microscopy and 3D digital images were taken with a three color video camera. The white bar represents a scale of 100 μm. Arrows indicate the size of the lesion in both untreated (vehicle) and treated (IA) mice retina. FIG. 2E shows that no significant effect was observed with the 20 μg dose. However, as shown in FIG. 2F, a significant difference (p =0.0443) of CNV size was observed between mice treated with 38 μg total rTnI containing the inhibitor of angiogenesis and those treated with vehicle.

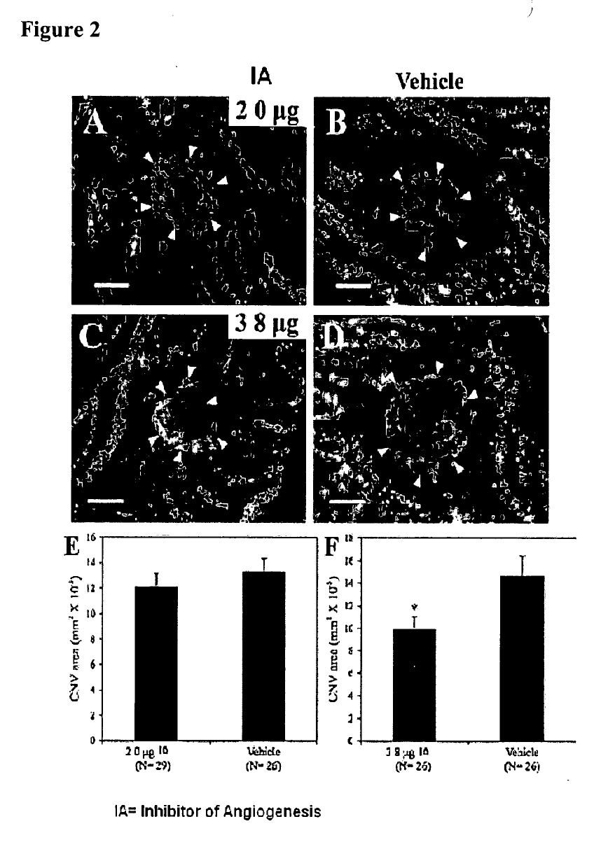

FIGS. 3A-3F are results showing the prevention of choroidal neovascularization by periocular injections of recombinant Troponin I administered daily for 14 days with a total dose of 53 μg and 28 μg, containing the inhibitor of angiogenesis. The CNV lesion was analyzed by fluorescence microscopy and 3D digital images were taken with a three color video digital camera. The white bar inside represents a scale of 100 um. Arrows indicate the size of the lesion induced by laser treatment in both untreated and treated mice eyes. FIG. 3E shows that when rTnI containing inhibitor of angiogenesis was administered at the total dosage of 28 μg no significant difference (p=0.6830) was observed in CNV size between mice retina treated with rTnI and those treated with vehicle. However, as shown in FIG. 3F, a significant difference (p=0.0002) of CNV size was observed between mice treated with 53 μg of total rTnI containing the inhibitor of angiogenesis and those treated with vehicle.

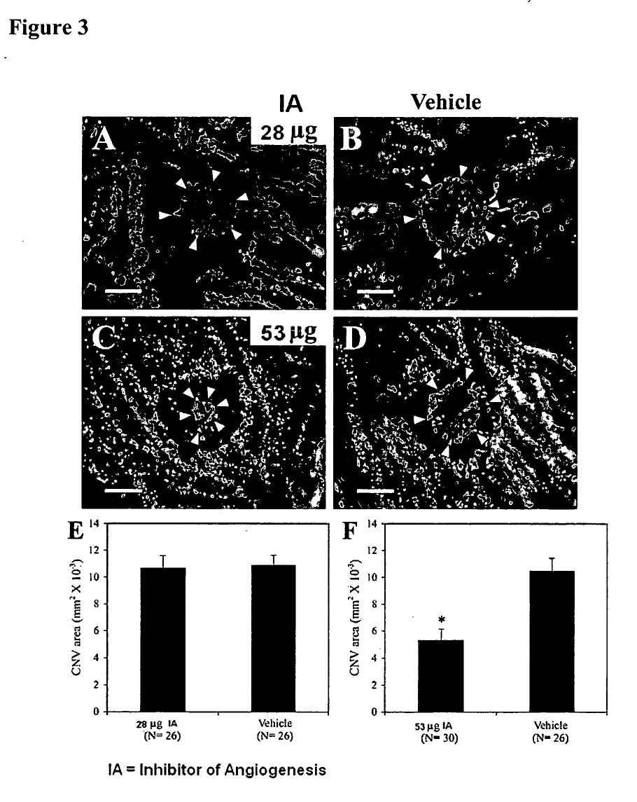

FIGS. 4A-4D show fluorescent microscope images from mice retina taken with a three color video digital camera. Regression of CNV is observed after administration of 38 μg of total rTnI containing the inhibitor of angiogenesis delivered by intravitreous injection. The white bar represents a scale of 100 μm. The p values were calculated with the Dunnett Method for multiple comparisons. Results show that *p value 3.172×10⁻¹⁰ of TpnI vs. the day 7 baseline is significant. Therefore, there was regression of CNV. The *† p value 3.013×10⁻⁷ of IA vs. the day 14 vehicle control is also significant, indicating that regression is not due to some nonspecific effect of injection.

All references cited herein are hereby incorporated by reference.

While the present invention has been described in conjunction with preferred embodiments, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

We claim:

1. A method of treating a condition characterized by neovascularization in a mammal, wherein the method comprises the step of administering to the mammal a therapeutically effective amount of an inhibitor of angiogenesis; said inhibitor of angiogenesis is derived from an extract of bacterial Escherichia coli and has a molecular weight ranging from about 7 to about 70 kDa.

2. The method of claim 1, wherein the condition is a cancer or ocular disease.

3. The method of claim 1, wherein the inhibitor of angiogenesis specifically inhibits mammalian endothelial cells.

4. The method of claim 1, wherein the inhibitor of angiogenesis specifically inhibits mammalian lung microvessel endothelial cells.

5. The method of claim 1, wherein the inhibitor of angiogenesis specifically inhibits mammalian choroidal vessel endothelial cells.

6. The method of claim 1, wherein the inhibitor of angiogenesis prevents neovascularization, prevents expansion of neovascularization or causes regression of neovascularization in retina.

7. The method of claim 1, wherein the inhibitor of angiogenesis prevents neovascularization, prevents expansion of neovascularization or causes regression of neovascularization in a tumor.

8. The method of claim 1, wherein the inhibitor of angiogenesis prevents the metastasis of tumor cells.

9. The method of claim 1, wherein the inhibitor of angiogenesis is selected from the group consisting of a peptide, protein, polysaccharide, lipopolysaccharide, oligonucleotide, DNA, pigment precursor, isoprenoid, small molecule, steroid, pyrimidine, and quinoline.

10. The method of claim 1, wherein the inhibitor of angiogenesis is murine lipoprotein.

11. The method of claim 1, wherein the inhibitor of angiogenesis is lumazine synthase.

12. The method of claim 1, wherein the inhibitor of angiogenesis is cold shock protein cspA or cspC.

13. The method of claim 1, wherein the inhibitor of angiogenesis is a lipopolysaccharide.
